(19) United States
(12) Patent Application Publication
(10) Pub. No.: US 2015/0164768 A1
(43) Pub. Date: Jun. 18, 2015

(54) Glycerin-in-Oil Emulsion

(71) Applicant: Avon Products, Inc., Suffern, NY (US)

(72) Inventor: Candice D. Novack, Monroe, NY (US)

(73) Assignee: Avon Products, Inc., Suffern, NY (US)

(21) Appl. No.: 14/108,835

(22) Filed: Dec. 17, 2013

(51) Int. Cl. A61K 8/73; A61K 8/34; A61Q 1/06; A61K 8/06

(52) U.S. Cl. CPC A61K 8/73; A61K 8/068; A61K 8/345; A61Q 1/06

(57) ABSTRACT

The present invention provides in one aspect, a glycerin-in-oil emulsion comprising (i) a continuous phase comprising one or more topically-acceptable oils; (ii) a discontinuous phase comprising glycerin and water; and (iii) an anionic polysaccharide in an amount sufficient to provide viscosity and cohesiveness to the discontinuous phase. In some embodiments, the emulsion further comprises an electrolyte such as magnesium sulfate in an amount sufficient to control droplet size of the discontinuous phase to be no more than 40 microns. Methods for making the emulsion are also provided.

### FIELD OF INVENTION

The present invention relates generally to methods and compositions for topical application to human integuments, including skin and lips. More specifically, the present invention relates to stable glycerin-in-oil emulsions and methods for making same.

### BACKGROUND

Emulsions are systems that consist of two or more liquid or solid phases that are partially or completely immiscible, with one phase being dispersed in the other in the form of droplets. Emulsions constitute an important product class in various industries including the food, chemical and pharmaceutical industries. Many cosmetics and personal care products, such as concealers, creams, lotions, and mascaras, are emulsions. Examples of common emulsions include water-in-oil, oil-in-water, silicone-in-water, and water-in-silicone emulsions. Either phase in an emulsion may further comprise a particulate phase, such as pigments.

However, emulsions present formulation challenges because the continuous and discontinuous phases are inherently immiscible and thus have a tendency to phase separate over time in order to minimize the thermodynamically unfavorable interaction between the two or more molecular species. Emulsions are known to undergo phase separation due to destabilization processes such as flocculation, coalescence, and Ostwald ripening. This instability can be exacerbated by temperature extremes. In order to be commercially viable an emulsion should exhibit sufficient stability to survive shipping and storage environments. For example, cosmetics are often shipped under conditions where they are exposed to temperatures higher and lower than standard room temperature (˜72° F.). Products must be stable at these temperature extremes so that they can be delivered to the customer in a form that is suitable for commercial sale. In addition, commercially acceptable cosmetics must also be shelf stable, such that they do not exhibit an inordinate degree of separation when stored for long periods of time, typically one, two to three years, and even longer in some instances. The tendency of the immiscible liquids or solids to separate out of the emulsion and coalesce frustrates these goals.

There has been interest in glycerin-in-oil and glycerin-in-silicone emulsions, particularly for skin and lip products, because glycerin is an effective humectant for retaining dermal moisture. Glycerin-in-oil emulsions are particularly unstable due to their chemical incompatibility and large density difference between the continuous and discontinuous phases. Even in the presence of compatibilizers, emulsifiers, etc., subjecting such emulsions to high temperature or alternating hot and cold temperatures (which is common during shipping and storage of cosmetic products) results in large scale phase separation which commonly manifests as the discontinuous (internal) phase leaching out of the continuous (external) phase. Such stability problems are not acceptable to consumers as the consumer may generally consider a product with separated phases or with leaching between phases to be unsatisfactory. Furthermore, instability may results in partial or complete loss of functionality and delivery of the composition. For example, if phase separation occurs, sweating (syneresis) of the internal phase may occur, resulting in uneven or messy consumer application.

A recent attempt to increase glycerin-in-oil stability is reported in Avon Products' U.S. Pub. No. 2011/0147259, the disclosure of which is hereby incorporated by reference in its entirety, which relates to the use of trihydroxystearin and 12-hydroxystearic acid to structure the continuous oil phase. The glycerin-in-oil emulsions are stable against repeated freeze-thaw cycles. While that approach successfully achieves stability by structuring the continuous oil phase, U.S. Pub. No. 2011/0147259 does not describe structuring the internal glycerin phase to stabilize glycerin-in-oil emulsions. Furthermore, in the case of solid or semi-solid emulsions, a formulator needs to consider more than Stokes settling of the internal phase. For example, in the case of emulsions which have a liquid internal phase and a solid external phase, one needs to balance the mechanical properties of the two phases.

It is therefore an object of the invention to provide compositions for application to human integuments, including, skin and lips, comprising glycerin-in-oil or glycerin-in-silicone emulsions having improved stability over time or improved stability when exposed to extreme temperatures.

### SUMMARY OF THE INVENTION

In accordance with the foregoing objectives and others, the present invention provides stabilized glycerin-in-oil emulsions and methods for stabilizing glycerin-in-oil emulsions. The emulsions are provided as compositions (e.g., cosmetic or therapeutic) for topical application to a human integument (e.g., hair, lashes, nails, skin, lips, etc.), particularly the skin of the face and lips.

The glycerin-in-oil emulsions of the invention are typically comprised of from about 25% to about 95% (w/w) continuous, external oil phase and from about 5% to about 75% (w/w) discontinuous, internal glycerin phase. The external phase may include any topically acceptable oil (e.g., ester oils, vegetable oils, hydrocarbon oils, silicone oils, etc.) and combinations of such oils in an amount from about 50% to about 100% by weight of the external phase. The external phase may optionally further comprise one or more waxes (e.g., microcrystalline wax, polyethylene wax, ozokerite wax, etc.) in an amount from about 0.1-30% by weight of the external phase. The emulsions may be in solid form, by which is meant they are freestanding, and may have a penetration value of at least 30 g. The discontinuous, internal phase comprises glycerin in an amount from about 10% to about 99% by weight (more typically, from about 55% to about 95% by weight), based on the weight of the discontinuous internal phase. The internal phase contains some amount of water but in a minor proportion (e.g., from about 0.1% to about 14% by weight based on the weight of the emulsion), and also an agent capable of structuring or thickening the glycerin phase (e.g., a polysaccharide thickener, such an anionic thickener, notably xanthan gum) in an amount effective to increase the viscosity of the glycerin phase (e.g., about 0.1-5% by weight based on the weight of the emulsion). An electrolyte (e.g., a water soluble salt such as NaCl, MgSO₄, etc.) can be optionally added in an amount effective (e.g., about 0.001-2% or 0.01-1% by weight of the emulsion) to modify the rheology, and in particular, to reduce the pituitous rheology of the thickened internal glycerin phase. An emulsifier may optionally be included as a component of either phase, typically in an amount from about 0.01% to about 6% by weight of the total emulsion. The emulsions are stabilized to provide greater lifetime for a retail product, either at room temperature or under the temperature extremes that the retail product may encounter. For example, the emulsions may be stable, meaning that there is no visible phase separation, for at least 10%, 20%, 50%, or at least 100% longer than an otherwise identical glycerin-in-oil emulsion in the absence of the agent capable of structuring or thickening the glycerin phase (e.g., xanthan gum), including after storage at about 49° C. (or even 60° C.) for an extended period of time such as 12 hours, one day, two days, three days, four days, five days, six days, one week, two weeks, three weeks, four weeks, or the like. The emulsions may have a maximum droplet size of the internal phase above or below 40 microns but typically will have a maximum droplet size less than about 30 microns, less than about 20 microns, or less than about 10 microns (e.g., from about 1-10 microns). The glycerin-in-oil emulsion may be incorporated into cosmetic compositions adapted for application to the lips, skin, or eye area, including, for example, lip products such as a lip cream, lip balm, lip gloss, medicated lip treatment, lip moisturizer, lip cosmetic, lip sunscreen, and lip flavorant.

The glycerin-in-oil emulsions may be prepared by a method comprising the steps of adding the discontinuous, internal glycerin phase comprising glycerin, water, a thickener (e.g., xanthan gum), and optionally an electrolyte (e.g., NaCl or MgSO₄), to the continuous, external oil phase while maintaining shear within a range suitable to reduce droplet size of the discontinuous phase to no more than about 40 microns (typically, applied shear of about 6-30 m/s). Prior to addition to the oil phase, the rheology of the internal phase may be characterized as having a complex viscosity of less than 2 Pa·s when subjected to an oscillatory stress of 100 Pa during an oscillatory stress sweep conducted at a temperature of 25° C. and an angular frequency of 1 Hz on a TA G2 Stress Controlled Rheometer with a parallel plate geometry gap of 500 micron.

These and other aspects of the present invention will become apparent to those skilled in the art after a reading of the following detailed description of the invention, including the appended claims.

### BRIEF DESCRIPTION OF THE DRAWINGS

FIG. 1 shows the storage modulus (G′) as a function of oscillating shear stress of the discontinuous, internal glycerin phase at different concentrations (0.1% or 0.05% by weight of entire emulsion) the anionic polysaccharide xanthan gum and with the optional addition of an electrolyte to the an anionic polysaccharide-containing discontinuous phase.

FIG. 2 shows pituitous rheology of samples utilizing glycerol and water (“A”); glycerol, water, and Xanthan gum (“B”); and glycerol, water, Xanthan gum and magnesium sulfate (“C”).

### DETAILED DESCRIPTION

All amounts provided in terms of weight percentage are relative to the entire emulsion (i.e., including both the internal, discontinuous and the external, continuous phases) unless otherwise stated. For the purposes of determining the weight percent of a component relative to the entire emulsion, any insoluble pigment phase is regarded as part of the oil phase. It will be understood that the total of all weight percentages in a given composition will not exceed 100%.

The term “consisting essentially of” is intended to include only those components that do not materially alter the basic and novel features of the inventive emulsions, including without limitation, the stability of the emulsion, the size of internal phase droplets, and/or the rheology of the internal phase or of the emulsion.

The term “anhydrous,” as used herein, refers to a composition to which no water is intentionally added but which may include trace amounts of moisture adsorbed or absorbed from the atmosphere. The term “substantially anhydrous,” as used herein, refers to a composition which may include up to 5% by weight water, but will typically comprise less than about 2.5% by weight water, or less than about 1% by weight water. In some embodiments, the internal phase of the present invention may be anhydrous or substantially anhydrous. In some embodiments, the glycerin from which the internal phase is prepared may also be anhydrous or substantially anhydrous.

As used herein, the term “oil” is intended to include silicone oils, unless otherwise noted. The term “oil” is intended to encompass volatile and/or nonvolatile oils. The terms “internal” and “discontinuous” phase are synonymous, as are the terms “external” and “continuous” phase. The terms “glycerin” and “glycerol” are synonymous and used interchangeably.

The compositions of the invention are useful for application to the human integumentary system, including, skin, lips, nails, hair, and other keratinous surfaces. As used herein, the term “keratinous surface” refers to keratin-containing portions of the human integumentary system, which includes, but is not limited to, skin, lips, hair (including eyebrows and eyelashes), and nails (toenails, fingernails, cuticles, etc.) of mammalians, preferably humans. A “keratin fiber” includes hair of the scalp, eyelashes, eyebrows, facial hair, and body hair such as hair of the arms, legs, etc.

The emulsions of the inventions are generally polyol-in-oil emulsions comprising a discontinuous, internal polyol (e.g., glycerin) phase and a continuous, external oil phase. The internal phase will typically comprise from about 5% to about 65% by weight of the entire emulsion. More typically, the internal phase will comprise from about 15% to about 45% by weight of the entire emulsion. The external phase will typically comprise from about 25% to about 90% by weight of the entire emulsion. More typically, the external phase will comprise from about 55% to about 85% by weight of the entire emulsion.

Suitable polyols for inclusion in the internal phase include, without limitation, C₂₋₆ polyols such as ethylene glycol, propylene glycol, butylene glycol, hexylene glycol, sorbitol, diethylene glycol, and glycerin. In some embodiments, the internal phase will comprise glycerin. In some embodiments, the internal phase will comprise glycerin in combination with one or more additional C₂₋₆ polyol components. In some cases, the internal phase will comprise glycerin as the major or predominant C₂₋₆ polyol component of the internal phase. Typically, the internal phase will comprise glycerin as the only C₂₋₆ polyol component of the internal phase. Ideally, the C₂₋₆ polyol will be one that is capable of provided a humectant benefit to the skin or lips.

To suppress Stokes settling of the internal phase, one can thicken or increase the viscosity of (“impart a structure to”) a liquid external phase, decrease the droplet size of the internal phase or balance the density ratio of the phases. In particular, to promote emulsion stability, it is conventional wisdom to increase the viscosity of the continuous (external) phase of the emulsion as reported in U.S. Pub. No. 2011/0147259, the disclosure of which is hereby incorporated by reference. However, it has been surprisingly found that structuring the internal phase (e.g., by using an anionic polysaccharide thickener), also improves emulsion stability, even in the absence of structuring the external phase.

Any structuring agent that can increase the viscosity of the internal glycerin phase is contemplated to be suitable. In some embodiments, a thickener (e.g., polysaccharide thickener) is used to structure the glycerin phase of the emulsion. In various embodiments, about 0.005%-5%, about 0.01-4%, about 0.05-2%, about 0.1-1%, or about 0.1-0.4% by weight thickener (e.g., polysaccharide thickener) can be used to effectively thicken the internal phase and thereby stabilize the emulsion.

Polysaccharide thickeners/structurants include, without limitation, natural vegetable gums, such as, Agar, alginic acid, sodium alginate, and Carrageenan, gum Arabic, gum ghatti, gum tragacanth, Karava gum, gaur gum, locust bean gum, beta-glucans, Chicle gum, Dammar gum, Glucomannan, Mastic gum, Psyllium seed husks, Spruce gum, Tara gum, Gellan gum, and xanthan gum; or synthetic cellulosic thickeners such as carboxymethyl cellulose, hydroxyethyl cellulose, hydroxypropyl cellulose, hydroxypropylmethyl cellulose, and the like.

In other embodiments, the thickener comprises, consists essentially of, or consists of a non-polysaccharide thickener. For example, polymers and copolymers of acrylic acid, including Acrylates Copolymer (INCI) are contemplated to be suitable. It has been found to be less desireable, however, to use acrylates based thickeners in embodiments where an electrolyte is added to the internal phase. Accordingly, in some embodiments, the compositions are substantially free of acrylate copolymer thickeners by which is meant that they are either absent or present in such a low level as to not have a material effect on the stability of the emulsion. In other embodiments, the compositions are substantially free of silica and inorganic clay thickeners (e.g., bentonite), by which is meant that they are either absent or present in such a low level as to not have a material effect on the stability of the emulsion. In one embodiment, the structuring agent comprises, consists essentially of, or consists of xanthan gum.

In some embodiments, the internal phase is comprised of: (1) from about 5% to about 100% (e.g., about 10-95%, about 20-85%, about 30-80%, or about 40-75%) by weight glycerin; (2) from about 0.001% to about 5% (e.g., about 0.005-3%, or about 0.01%-2%, or about 0.05-1.5%, or about 0.1-1%) by weight, based on the total weight of the emulsion, of a thickener capable of increasing the viscosity of the glycerin phase (e.g., an anionic polysaccharide, such as xanthan gum); (3) from about 1% to about 14% (e.g., about 1-12%, or about 2-10%, or about 3-8%, or about 3-5%) by weight, based on the total weight of the emulsion, water; (4) optionally, from about 0.001% to about 5% (e.g., about 0.01-3%, or about 0.05-2.5%, or about 0.1-2%) by weight of an electrolyte, such as a salt of a Group IA or Group IIA metal (e.g., NaCl, CaCl₂, MgCl₂, MgSO₄, etc.) soluble in the internal phase; (5) optionally, from about 0.1 to about 15% by weight of an emulsifier, and (6) optionally, from about 0.001% to about 30% by weight of additional ingredients soluble or dispersible in the internal phase, including without limitation, water soluble or dispersible film forming polymers, additional rheology modifiers, stabilizers, dispersants, humectants (e.g., additional C₃₋₂₄ polyols, such as propylene glycol, sugar alcohols, sorbitol, xylitol, butylene glycol, polyglycerol, and the like), active ingredients (e.g., collagenase inhibitors, elastase inhibitors, collagen stimulators, depigmenting agents, desquamating agents, etc.), antimicrobials, preservatives, pH adjusters, colorants, fragrances, flavorants and the like.

In some embodiments, incorporating water into the discontinuous phase can counteract the hygroscopic nature of polar solvent (e.g., polyol) present in the emulsion. For example, about 0.1-10%, about 0.5-8%, about 1-5%, or about 2-4% by weight water may be present. The water may be intentionally added to the discontinuous phase, or it may be present in the polyol phase due to the hygroscopic nature of the polyol, or a combination of the two. In one embodiment, water is added to the internal phase. In another embodiment, the water added to the internal phase is distilled water.

In some embodiments, a water soluble salt of (i) a cation selected from Group IA metal, Group IIA metals, ammonium, or a quaternary amine, with (ii) an anion selected from halide, sulfate, sulfite, carbonate, bicarbonate, and phosphate), may be added to the internal phase. Suitable electrolytes include, without limitation, salts such as sodium chloride, potassium chloride, calcium chloride, magnesium chloride, sodium sulfate, calcium sulfate, potassium sulfate, magnesium sulfate, sodium carbonate, sodium bicarbonate, ammonium sulfate, mono- di- and tri-sodium phosphate, mono- di- and tri-potassium phosphate, and various other salts known in the art) can be added to the internal phase, including by reducing the pituitous rheology (e.g., stringiness) of the internal phase and the emulsion, thereby allowing easier break up of the internal phase during processing. As a result, the addition of salt allows smaller droplets of the internal phase to be formed. In general, small droplets are (e.g., less than 40 microns in diameter) are preferred, as larger droplets may be visually less appealing to the consumer and tend to be less stable. Thus, inclusion of a salt can be used to control droplet size. For example, a salt in an amount sufficient to control droplet size of the discontinuous phase can added to the emulsion of the present invention during processing to provide a discontinuous phase having a maximum droplet size of 40 microns. In certain embodiments, the discontinuous phase can have a maximum droplet size of 1-10 microns. If present, the amount of salt added may be from about 0.001-2.5%, about 0.01-1.5%, about 0.10-1%, or about 0.20-0.50% by weight.

In some embodiments, the rheology of the internal phase may be characterized as having a complex viscosity of less than 2 Pa·s when subjected to an oscillatory stress of 100 Pa during an oscillatory stress sweep conducted at a temperature of 25° C. and an angular frequency of 1 Hz on a TA G2 Stress Controlled Rheometer with a parallel plate geometry gap of 500 micron.

In one embodiment, the internal phase (excluding any particulate phase dispersed within the internal phase) comprises, consists essentially of, or consists of from 90-100% by weight glycerin, about 1-10% water, about 0.01-1.5% Xanthan gum, and from 0.001-2.5%, magnesium sulfate, and optionally may further include from 0.01-10% by weight of additional active and inactive ingredients, including without limitation, water soluble or dispersible film forming polymers, additional rheology modifiers, stabilizers, dispersants, humectants (e.g., additional C₃₋₂₄ polyols, such as propylene glycol, sugar alcohols, sorbitol, xylitol, butylene glycol, polyglycerol, and the like), active ingredients (e.g., collagenase inhibitors, elastase inhibitors, collagen stimulators, depigmenting agents, desquamating agents, etc.), antimicrobials, preservatives, pH adjusters, colorants, and fragrances.

The continuous phase may comprise any suitable oils for emulsions, including, without limitation, vegetable oils; fatty acid esters; fatty alcohols; isoparaffins such as isododecane and isoeicosane; hydrocarbon oils such as mineral oil, petrolatum, and polyisobutene; polyolefins and hydrogenated analogs thereof (e.g., hydrogenate polyisobutene); natural or synthetic waxes; silicone oils such as dimethicones, cyclic silicones, and polysiloxanes; and the like.

Suitable ester oils include fatty acid esters. Special mention may be made of those esters commonly used as emollients in cosmetic formulations. Such esters will typically be the etherification product of an acid of the form R₄(COOH)₁₋₂ with an alcohol of the form R₅(OH)₁₋₃ where R₄ and R₅ are each independently linear, branched, or cyclic hydrocarbon groups, optionally containing unsaturated bonds (e.g., from 1-6 or 1-3 or 1), and having from 1 to 30 (e.g., 6-30 or 8-30, or 12-30, or 16-30) carbon atoms, optionally substituted with one or more functionalities including hydroxyl, oxa, oxo, and the like. Preferably, at least one of R₄ and R₅ comprises at least 8, or at least 10, or at least 12, or at least 16 or at least 18 carbon atoms, such that the ester comprises at least one fatty chain. The esters defined above will include, without limitation, the esters of mono-acids with mono-alcohols, mono-acids with diols and triols, di-acids with mono-alcohols, and tri-acids with mono-alcohols.

Suitable fatty acid esters include, without limitation, butyl acetate, butyl isostearate, butyl oleate, butyl octyl oleate, cetyl palmitate, ceyl octanoate, cetyl laurate, cetyl lactate, cetyl isononanoate, cetyl stearate, diisostearyl fumarate, diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisopropyl dimerate, triisostearyl trilinoleate, octodecyl stearoyl stearate, hexyl laurate, hexadecyl isostearate, hexydecyl laurate, hexyldecyl octanoate, hexyldecyl oleate, hexyldecyl palmitate, hexyldecyl stearate, isononyl isononanaote, isostearyl isononate, isohexyl neopentanoate, isohexadecyl stearate, isopropyl isostearate, n-propyl myristate, isopropyl myristate, n-propyl palmitate, isopropyl palmitate, hexacosanyl palmitate, lauryl lactate, octacosanyl palmitate, propylene glycol monolaurate, triacontanyl palmitate, dotriacontanyl palmitate, tetratriacontanyl palmitate, hexacosanyl stearate, octacosanyl stearate, triacontanyl stearate, dotriacontanyl stearate, stearyl lactate, stearyl octanoate, stearyl heptanoate, stearyl stearate, tetratriacontanyl stearate, triarachidin, tributyl citrate, triisostearyl citrate, tri-C₁₂₋₁₃-alkyl citrate, tricaprylin, tricaprylyl citrate, tridecyl behenate, trioctyldodecyl citrate, tridecyl cocoate, tridecyl isononanoate, glyceryl monoricinoleate, 2-octyldecyl palmitate, 2-octyldodecyl myristate or lactate, di(2-ethylhexyl)succinate, tocopheryl acetate, and the like.

Other suitable esters include those wherein R₅ comprises a polyglycol of the form H—(O—CHR*—CHR*)ₙ— wherein R* is independently selected from hydrogen or straight chain C₁₋₁₂ alkyl, including methyl and ethyl, as exemplified by polyethylene glycol monolaurate.

Salicylates and benzoates are also contemplated to be useful esters in the practice of the invention. Suitable salicylates and benzoates include esters of salicylic acid or benzoic acid with an alcohol of the form R₆OH where R₆ is a linear, branched, or cyclic hydrocarbon group, optionally containing unsaturated bonds (e.g., one, two, or three unsaturated bonds), and having from 1 to 30 carbon atoms, typically from 6 to 22 carbon atoms, and more typically from 12 to 15 carbon atoms. Suitable salicylates include, for example, octyl salicylate and hexyldodecyl salicylate, and benzoate esters including C₁₂₋₁₅ alkyl benzoate, isostearyl benzoate, hexyldecyl benzoate, benzyl benzoate, and the like.

Other suitable esters include, without limitation, polyglyceryl diisostearate/IPDI copolymer, triisostearoyl polyglyceryl-3 dimer dilinoleate, polyglycerol esters of fatty acids, and lanolin, to name but a few.

The oil may also comprise a volatile or non-volatile silicone oil. Suitable silicone oils include linear or cyclic silicones such as polyalkyl- or polyarylsiloxanes, optionally comprising alkyl or alkoxy groups having from 1 to 10 carbon atoms. Representative silicone oils include, for example, caprylyl methicone, cyclomethicone, cyclopentasiloxane decamethylcyclopentasiloxane, decamethyltetrasiloxane, diphenyl dimethicone, dodecamethylcyclohexasiloxane, dodecamethylpentasiloxane, heptamethylhexyltrisiloxane, heptamethyloctyltrisiloxane, hexamethyldisiloxane, methicone, methyl-phenyl polysiloxane, octamethylcyclotetrasiloxane, octamethyltrisiloxane, perfluorononyl dimethicone, polydimethylsiloxanes, and combinations thereof. The silicone oil will typically, but not necessarily, have a viscosity of between about 5 and about 3,000 centistokes (cSt), preferably between 50 and 1,000 cSt measured at 25° C.

In one embodiment, the silicone oil comprises phenyl groups, as is the case for a silicone oil such as methylphenylpolysiloxane (INCI name diphenyl dimethicone), commercially available from Shin Etsu Chemical Co under the name including F-5W, KF-54 and KF-56. Diphenyl dimethicones have good organic compatibility and may impart film-forming characteristics to the product. Further, the presence of phenyl groups increases the refractive index of the silicone oil and thus may contribute to high gloss of product if desired. In one embodiment, the silicone oil will have a refractive index of at least 1.3, preferably at least 1.4, more preferably at least 1.45, and more preferred still at least 1.5, when measured at 25° C. Another suitable phenyl-functionalized silicone oil has the INCI name phenyltrimethicone and is sold under the trade name DC 556 by Dow Corning. DC 556 has a refractive index of about 1.46. In one embodiment, the silicone oil is a fluorinated silicone, such as a perfluorinated silicone (i.e., fluorosilicones). Fluorosilicones are advantageously both hydrophobic and oleophobic and thus contribute to a desirable slip and feel of the product. Fluorosilicones also impart long-wearing characteristics to a lip product. Fluorosilicones can be gelled with behenyl behenate if desired. One suitable fluorosilicone is a fluorinated organofunctional silicone fluid having the INCI name perfluorononyl dimethicone. Perfluorononyl dimethicone is commercially available from Pheonix Chemical under the trade name PECOSIL®.

The compositions may also comprise hydrocarbon oils. Exemplary hydrocarbon oils are straight or branched chain paraffinic hydrocarbons having from 5 to 80 carbon atoms, typically from 8 to 40 carbon atoms, and more typically from 10 to 16 carbon atoms, including but not limited to, pentane, hexane, heptane, octane, nonane, decane, undecane, dodecane, tetradecane, tridecane, and the like. Some useful hydrocarbon oils are highly branched aliphatic hydrocarbons, including C₈₋₉ isoparaffins, C₉₋₁₁ isoparaffins, C₁₂ isoparaffin, C₂₀₋₄₀ isoparaffins and the like. Special mention may be made of the isoparaffins having the INCI names isohexadecane, isoeicosane, and isododecane (IDD).

Also suitable as hydrocarbon oils are poly-alpha-olefins, typically having greater than 20 carbon atoms, including (optionally hydrogenated) C₂₄₋₂₈ olefins, C₃₀₋₄₅ olefins, polyisobutene, hydrogenated polyisobutene, hydrogenated polydecene, polybutene, hydrogenated polycyclopentane, mineral oil, pentahydrosqualene, squalene, squalane, and the like. The hydrocarbon oil may also comprise higher fatty alcohols, such as oleyl alcohol, octyldodecanol, and the like.

Other suitable oils include without limitation castor oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, avocado oil, camellia oil, macadamia nut oil, turtle oil, mink oil, soybean oil, grape seed oil, sesame oil, maize oil, rapeseed oil, sunflower oil, cottonseed oil, jojoba oil, peanut oil, olive oil, and combinations thereof.

Any one of the foregoing ester oils, silicone oils, and hydrocarbon oils are contemplated to be useful in the practice of the invention. Accordingly, in one embodiment, the compositions comprise at least one oil selected from the ester oils, silicone oils, and hydrocarbon oils described above. In another embodiment, the compositions comprise two or more oils selected from the ester oils, silicone oils, and hydrocarbon oils described above. In yet another embodiment, the compositions will comprise at least one ester, at least one silicone oil, and at least one hydrocarbon oil from the list above. Because the ester oils described herein function as emollients, it may be advantageous for the compositions comprise at least one ester oil, and may optionally comprise at least one additional oil selected from hydrocarbon oils, silicone oils, and combinations thereof.

In one embodiment, the continuous phase includes lanolin, typically low odor lanolin. In one embodiment, the oil phase comprises lanolin in an amount of 1-100% by weight of the oil phase. More typically, the oil phase will comprises lanolin in an amount from about 5-15%, or from about 15-25%, or from about 25-35%, or from about 35-45%, or from about 45-55%, or from about 55-65%, or from about 65-75%, or from about 75-85%, or from about 85-95%, or from about 95-100% by weight of the oil phase. In one embodiment, the oil phase comprises lanolin in at least about 20% by weight of the total emulsion phase. In another embodiment, the oil phase comprises lanolin in at least about 25% by weight of the total emulsion. In yet another embodiment, the oil phase comprises lanolin in an amount between about 20-50% or between about 25-35% by weight of the total emulsion.

In some embodiments, the oil phase can include one or more waxes. Waxes may impart body to the emulsion so that the emulsion has the physical form of a semi-solid or solid. As used herein, the term “solid” is intended to refer to a composition that is self-supporting and capable of being molded into a free-standing stick (e.g., a lip stick). In some embodiments, the waxes are present in an amount sufficient to make the emulsion a solid emulsion. For example, the solid emulsion can have a hardness of at least 30 g. The composition typically has hardness at room temperature of at least 40 g. In one embodiment, the composition may have a substantially greater hardness, between about 100 and about 300 g. The hardness of an emulsion may be measured on a Texture Analyzer Model QTS-25 equipped with a 4 mm stainless steel probe (TA-24), as described in Avon's U.S. Pat. No. 8,580,283, the disclosure of which is hereby incorporated by reference.

As used herein, the term “penetration” refers to the relative hardness of the wax at a specified temperature. Penetration may be measured using ASTM-D1321-02a, incorporated by reference herein. A higher penetration value indicates a harder wax.

The waxes may be natural, mineral and/or synthetic waxes. Natural waxes include those of animal origin (e.g., beeswax, spermaceti, lanolin, and shellac wax) and those of vegetable origin (e.g., carnauba, candelilla, bayberry, and sugarcane wax). Mineral waxes include, without limitation ozokerite, ceresin, montan, paraffin, microcrystalline, petroleum, and petrolatum waxes. Synthetic waxes include, for example, polyethylene glycols such as PEG-18, PEG-20, PEG-32, PEG-75, PEG-90, PEG-100, and PEG-180 which are sold under the tradename CARBOWAX® (The Dow Chemical Company). Mention may be made of the polyethylene glycol wax CARBOWAX 1000 which has a molecular weight range of 950 to 1,050 and a melting point of about 38° C., CARBOWAX 1450 which has a molecular weight range of about 1,305 to 1,595 and a melting point of about 56° C., CARBOWAX 3350 which has a molecular weight range of 3,015 to 3,685 and a melting point of about 56° C., and CARBOWAX 8000 which has a molecular weight range of 7,000 to 9,000 and a melting point of about 61° C.

Synthetic waxes also include Fischer Tropsch (FT) waxes and polyolefin waxes, such as ethylene homopolymers, ethylene-propylene copolymers, and ethylene-hexene copolymers. Representative ethylene homopolymer waxes are commercially available under the tradename POLYWAX® Polyethylene (Baker Hughes Incorporated) with melting points ranging from 80° C. to 132° C. Commercially available ethylene-α-olefin copolymer waxes include those sold under the tradename PETROLITE® Copolymers (Baker Hughes Incorporated) with melting points ranging from 95° C. to 115° C.

In one embodiment, the emulsion includes, in the oil phase, at least one wax selected from arcawax (N,N′-ethylenebisstearamide), microcrystalline wax, linear polyethylene wax, stearone (18-pentatriacontanone), castor wax, montan wax, lignite wax, ouricouri wax, carnauba wax, rice bran wax, shellac wax, esparto wax, ozokerite wax, jojoba wax, candelilla wax, ceresin wax, beeswax, castor wax, sugarcane wax, stearyl alcohol, hard tallow, cetyl alcohol, petrolatum, glyceryl monostearate, Japan wax, silicone wax, paraffin wax, lanolin wax, lanolin alcohol, bayberry wax, cetyl palmitate, illipe butter, cocoa butter, and ethylene glycol di- or tri-esters of C₁₈₋₃₆ fatty acids.

The amount of wax, if present, will typically be less than about 2% (e.g., 0.1-2%) by weight of the emulsion if the emulsion is a liquid or if clarity is desired. The amount of wax, if present, will typically be greater than about 10% (e.g., 10-20%) by weight of the emulsion if the emulsion is a semisolid or solid or if clarity is not a concern. In some embodiments, the emulsion may comprise wax from about 5% to about 25% (or about 1-20% or about 12-18%) by weight based on the weight of the emulsion, particularly in embodiments formulated as lip sticks.

In one embodiment, the emulsion includes, in the oil phase, from 0.1-2% or 2-5% or 5-10% or 10-15% or 15-20% by weight of at least one wax selected from microcrystalline wax, ozokerite wax, and polyethylene wax. In one embodiment, the emulsion includes, in the oil phase, microcrystalline wax within the foregoing amounts. In one embodiment, the emulsion includes, in the oil phase, ozokerite wax within the foregoing amounts. In one embodiment, the emulsion includes, in the oil phase, polyethylene wax within the foregoing amounts.

Typically, emulsions according to the invention further comprise one or more emulsifiers. For example, the one or more emulsifiers may be present in a total range from about 0.01% to about 10.0% by weight of the emulsion. In some embodiments, the total amount of emulsifier ranges from about 0.1% to about 6.0% be weight, or from about 0.5% to about 4.0% by weight. Emulsifiers having a lower HLB value may be suitable for use in glycerin-in-oil emulsions. For example, such emulsifiers may have a low HLB of below 10, or below 8.5. In certain embodiments, HLB values are between 2 and 5. In one embodiment, one or more low HLB emulsifiers is used in combination with a higher HLB emulsifier. Examples of emulsifiers include polyglyceryl compounds such as polyglyceryl-6-polyricinoleate, polyglyceryl pentaoleate, polyglyceryl-isostearate, and polyglyceryl-2-diisostearate; glycerol esters such as glycerol monostearate or glycerol monooleate; phospholipids and phosphate esters such as lecithin and trilaureth-4-phosphate (available under the tradename HOSTAPHAT®KL-340-D); sorbitan-containing esters (including SPAN® esters) such as sorbitan laurate, sorbitan oleate, sorbitan stearate, or sorbitan sesquioleate; polyoxyethylene phenols such as polyoxyethylene octyl phenol; polyoxyethylene ethers such as polyoxyethylene cetyl ether and polyoxyethylene stearyl ether; polyethylene glycol emulsifiers such as PEG-30-polyhydroxystearate or alkylpolyethylene glycols; polypropylene glycol emulsifiers such as PPG-6-laureth-3; dimethicone polyols and polysiloxane emulsifiers; and the like. Combinations of emulsifiers, such as the combination of lecithin and sorbitan, are envisioned. Additional emulsifiers are provided in the INCI Ingredient Dictionary and Handbook, 12th Edition, 2008, the disclosure of which is hereby incorporated by reference.

Additional components may be incorporated for various functional purposes as is customary in the cosmetic arts into the internal phase, the external phase, or as a particulate phase. However, while additional components consistent to formulate the above cosmetic compositions may be included, the inclusion of additional ingredients is limited to those ingredients in amounts which do not interfere with the formation or stability of a polyol-in-oil (e.g., glycerin-in-oil) emulsion.

When formulated as cosmetic compositions for topical application, the emulsions will typically include additional components optionally distributed in either or both phases of the emulsion. Such components may be selected from the group consisting of film-formers, pigments, waxes, emollients, moisturizers, preservatives, flavorants, antioxidants, botanicals, and mixtures thereof. Particular mention may be made of highly purified botanical extracts or synthetic agents which may have wound-healing, anti-inflammatory, or other benefits useful for treating the skin or lips. Additional embodiments may include antioxidants such as tocopherol. The compositions may include one or more film-formers to increase the substantivity of the product. In certain embodiments, compositions according to the invention provide high moisturization readings upon topical application due to the presence of high levels of glycerin while also achieving consumer acceptance due to increased stability.

For example, in addition to the polysaccharide thickener, the composition may comprise other thickeners known in the art, such as vegetable gums, carboxymethyl cellulose, silica, acrylic acid polymers, clays, such as hectorites, bentonites, hydrated magnesium and aluminium silicates, or calcium silicates, or the like. When present, these additional thickeners will comprise from about 0.1% to about 15% by weight of the composition, more typically from about 1% to about 5% by weight of the composition.

Film formers, including film forming polymers, may also be employed. The term film-forming polymer may be understood to indicate a polymer which is capable, by itself or in the presence of at least one auxiliary film-forming agent, of forming a continuous film which adheres to a surface and functions as a binder for the particulate material. Polymeric film formers include, without limitation, acrylic polymers or co-polymers, (meth)acrylates, alkyl(meth)acrylates, polyolefins, polyvinyls, polacrylates, polyurethanes, silicones, polyamides, polyethers, polyesters, fluoropolymers, polyethers, polyacetates, polycarbonates, polyamides, polyimides, rubbers, epoxies, formaldehyde resins, organosiloxanes, dimethicones, amodimethicones, dimethiconols, methicones, silicone acrylates, polyurethane silicones copolymers, cellulosics, polysaccharides, polyquaterniums, and the like. Suitable film formers include those listed in the Cosmetic Ingredient Dictionary (INCI and Handbook, 12th Edition (2008), the disclosure of which is hereby incorporated by reference.

The cosmetic compositions of the invention may optionally include one or more agents that provide or enhance shine. Shine enhancing agents will typically have a refractive index greater than about 1.4, preferably greater than about 1.5 when measured as a film at 25° C. Suitable shine enhancing agents include without limitation, polyols, fatty esters, silicone phenylpropyldimethylsiloxysilicate, polybutene, polyisobutene, hydrogenated polyisobutene, hydrogenated polycyclopentadiene, propyl phenyl silsesquioxane resins; lauryl methicone copolyol, perfluorononyl dimethicone, dimethicone/trisiloxane, methyl trimethicone, and combinations thereof. In one embodiment, the composition will comprise a shine-enhancing agent in an amount from about 0.1% to about 10% by weight, more typically from about 1% to about 5% by weight, based on the total weight of the composition.

Particulate materials may be added for ultraviolet (UV) light absorption or scattering, such as titanium dioxide and zinc oxide particulates, or for aesthetic characteristics, such as color (e.g., pigments and lakes), pearlescence (e.g., mica, bismuth oxychloride, etc.), or the like. If present, the particulate phase will comprise from about 0.1 to about 25% of the weight of the entire composition. More typically, the particulate phase will comprise from about 2.5% to about 15% by weight of the entire composition.

The particulates may comprise colorants, including pigments and lakes. As used herein, the term “pigments” embraces lakes and fillers such as talc, calcium carbonate, etc. Exemplary inorganic pigments include, but are not limited to, inorganic oxides and hydroxides such as magnesium oxide, magnesium hydroxide, calcium oxide, calcium hydroxides, aluminum oxide, aluminum hydroxide, iron oxides (α-Fe₂O₃, γ-Fe₂O₃, Fe₃O₄, FeO) and iron hydroxides including red iron oxide, yellow iron oxide and black iron oxide, titanium dioxide, titanium lower oxides, zirconium oxides, chromium oxides, chromium hydroxides, manganese oxides, manganese hydroxides, cobalt oxides, cobalt hydroxides, cerium oxides, cerium hydroxides, nickel oxides, nickel hydroxides, zinc oxides and zinc hydroxides and composite oxides and composite hydroxides such as iron titanate, cobalt titanate and cobalt aluminate and the like. Preferably, the inorganic oxide particles may be selected from silica, alumina, zinc oxide, iron oxide and titanium dioxide particles, and mixtures thereof. In one embodiment, the pigments have a particle size from 5 nm to 500 microns, or from 5 nm to 250 microns, or from 10 nm to 100 microns. In some embodiments, the particle size (median) will be less than bout 5 microns or less than 1 micron.

Additional exemplary color additive lakes include, for example: D&C Red No. 19 (e.g., CI 45170, CI 73360 or CI 45430); D&C Red No. 9 (CI 15585); D&C Red No. 21 (CI 45380); D&C Orange No. 4 (CI 15510); D&C Orange No. 5 (CI 45370); D&C Red No. 27 (CI 45410); D&C Red No. 13 (CI 15630); D&C Red No. 7 (CI 15850:1); D&C Red No. 6 (CI 15850:2); D&C Yellow No. 5 (CI 19140); D&C Red No. 36 (CI 12085); D&C Orange No. 10 (CI 45475); D&C Yellow No. 19 (CI 15985); FD&C Red #40 (CI#16035); FD&C Blue #1 (CI#42090); FD&C Yellow #5 (CI#19140); or any combinations thereof.

Suitable, cosmetic particulates include, without limitation, methylsilsesquioxane resin microspheres, for example, TOSPEARL™ 145A, (Toshiba Silicone); particles of polymethylsilsesquioxane sold under the name TOSPEARL™ 150 KA (Kobo); microspheres of polymethylmethacrylates, for example, MICROPEARL™ 100 (Seppic); spherical particles of polymethylmethacrylate, such as those sold under the name TECEEPOLYMER™ MB-8CA (KOBO)); particles of VinylDimethicorte/Methicone Silsesquioxane Crosspolymer sold under the name KSP™ 105 (Shin-Etsu); the spherical particles of crosslinked polydimethylsiloxanes, for example, TREFIL™ E 506C or TREHL™ E 505C (Dow Corning Toray Silicone); spherical particles of polyamide, for example, nylon-12, and ORGASOL™ 2002D Nat C05 (Atochem); polystyrene microspheres, for example Dyno Particles, sold under the name DYNOSPHERES™, and ethylene acrylate copolymer, sold under the name FLOBEAD™ EA209 (Kobo); aluminum starch octenylsuccinate, for example DRY FLO™ (National Starch); microspheres of polyethylene, for example MKROTEIENE™ FN510-00 (Equistar), spherical particles of PTFE, available under the name FLUOROPURE™ 109 C (Shamrock) or MICROSLIP™ 519 (Presperse); silicone resin, polymethylsasesquioxane silicone polymer, Polysilicones, including without limitation, Polysilicone-1, Polysilicone-2, Polysilicone-3, Polysilicone-4, Polysilicone-5, Polysilicone-6, Polysilicone-7, Polysilicone-8, Polysilicone-10, Polysilicone-11, Polysilicone-12, Polysilicone-13, Polysilicone-14, Polysilicone-15, Polysilicone-16, Polysilicone-17, Polysilicone-18, and Polysilicone-19; Dimethicone/Divinyidimethicone/Silsesquioxane Crosspolymer (available under the trade name GRANSIL EPSQ from Grant Industries); dimethicone/silsesquioxane copolymer (available under the trade name SILDERM EPSQ from Active Concepts); platelet shaped powder made from N-lauroyl lysine, available under the name AMIHOPE™ LL (Ajinomoto), and mixtures thereof, to name a few. Other suitable particulates include the particulate silicon wax sold under the trade name TEGOTOP™ 105 (Degussa/Goldschmidt Chemical Corporation) and the particulate vinyl polymer sold under the name MINCOR™ 300 (BASF).

The composition may comprise one or more preservatives or antimicrobial agents, such as methyl, ethyl, or propyl paraben, and so on, in amounts ranging from about 0.0001-5 wt % by weight of the total composition. The compositions may have other ingredients such as one or more anesthetics, anti-allergenics, antifungals, anti-inflammatories, antimicrobials, antiseptics, chelating agents, emollients, emulsifiers, fragrances, humectants, lubricants, masking agents, medicaments, moisturizers, pH adjusters, preservatives, protectants, soothing agents, stabilizers, sunscreens, surfactants, thickeners, viscosifiers, vitamins, or any combinations thereof.

In one embodiment, the emulsions according to the invention are provided as products for application to the lips. Such lip products may include lip cream, lip balm, lip gloss, medicated lip treatment, lip moisturizer, lip cosmetic, lip sunscreen, and lip flavorant. In one embodiment, the lip product is a creamy, flowable lip product. In certain embodiments, products according to the invention may have the consistency of a semi-viscous liquid or paste. In other embodiments, the product is a lip stick.

When formulated as lip products, the emulsions according to the invention may be packaged in a re-closeable container. Such containers may include an enclosure or chamber charged with the emulsion formulated as a cosmetic composition and a cap removably attached to the container or reversibly configured on the container. In one embodiment, a cap may be attached to a squeezable enclosure (e.g., formed of a pliant plastic material) such that the cap can be removed from the orifice of the squeezable enclosure and replaced upon completion of dispensing of the composition. A cap may be attached to the body of a squeezable enclosure (e.g., by screw threads, a snap fit, or the like), to facilitate re-sealing the squeezable enclosure for storage between uses. In one embodiment, the cap is reversibly attached to the container for sealing the contents when in a closed position and for permitting the contents of the container to be dispensed when in an open position. Various containers are envisioned, including without limitation click pens, barrel dispensers, pumps, air-less pumps, pressurized packages, hand-squeezed containers, a cosmetic applicator, and the like.

In other embodiments, the emulsions may be in the form of skin care emulsions (lotions, creams, gels, etc.), color cosmetics, mascaras, eye shadows, lip color, lip liner, foundation, concealer, make up remover, sunscreen, deodorants, to name a few.

Exemplary ranges of composition ingredients are provided in Table 1 (percentages are listed as weight percentage of the entire emulsion including particulates) for a cosmetic composition, such as a lipstick. It should be noted that some components are optional.

TABLE 1

| | |
|---|---|
| % (w/w) | Ingredient |

Discontinuous (Internal) Phase

| | |
|---|---|
| 2-40% | Glycerin |
| 1-10% | Water |
| 0.01-5% | Polysaccharide Thickener |
| 0.00-2% | Electrolyte |
| 0-0.001-10% | Additional cosmetic ingredients |

Continuous (External) Phase

| | |
|---|---|
| 2-30% | Wax |
| 1-60% | Oils |
| 0.1-10% | Emulsifiers |
| 0-0.1-20% | Additional cosmetic ingredients |
| 1-30% | Pigments, lakes, fillers |

It will be understood that the sum of all weight percentage of components in Table 1 does not exceed 100%. The additional cosmetic ingredients include any active and inactive ingredients known in the art, including those described above. The electrolyte in this embodiment may be magnesium sulfate, the polysaccharide thickener may comprise xanthan gum, and the oil may comprise lanolin.

In some embodiments, the stabilized emulsions of the invention are stable on standing at room temperature (˜25° C.) for one week, two weeks, three weeks, four weeks, or even longer. In some embodiments, the stabilized emulsions of the invention are stable after heating to about 49° C. for one week, two weeks, three weeks, four weeks, or even longer. Stability can be measured visually by a lack or phase separation or syneresis.

In another embodiment, an emulsion is produced wherein the electrolyte is magnesium sulfate.

In another embodiment, an emulsion is produced wherein water comprises from about 3% to about 14% by weight of the entire emulsion.

In another embodiment, an emulsion is produced wherein water comprises from about 3% to about 5% by weight of the entire emulsion.

In another embodiment, an emulsion is produced wherein a polysaccharide comprises from about 0.01% to about 5% by weight of the entire emulsion.

In another embodiment, an emulsion is produced wherein a polysaccharide comprises from about 0.1% to about 1% by weight of the entire emulsion.

In another embodiment, an emulsion is produced wherein an anionic polysaccharide is Xanthan gum.

In another embodiment, an emulsion is produced wherein lanolin is present in a range of from about 25% to about 40% of the entire composition.

In another embodiment, lanolin is present as an oil constituent of the oil phase.

In one embodiment, the composition is intended for use as a non-therapeutic treatment.

In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, §201(i).

### Examples

### Example 1

### Methods for Making Emulsion

Formulas 1-6 described hereunder were made according to the following method:
Heat and melt the waxes, oils and emulsifiers (oil phase) to 85-90° C.
Stir continuously and add the colorants.
Mix at high speed until well blended.
Separately, heat and mix with high shear the glycerin, water and xanthan gum (glycerin phase).
Slowly add the glycerin phase to the wax phase while maintaining high shear within the processing range described below.
Shear for 30 minutes.

The high shear environment is ideally closely controlled during processing. Shearing at a minimum of 8 m/s and a maximum of 10 m/s is one acceptable range, although it will be understood that the tolerable shear will be somewhat dependent on the type of mixer or mill that is used and deviations from these values are to be expected. It is within the skill in the art to determine suitable shear for forming the emulsions of the invention, and in particular those skilled in the art will be guided by the principle that the magnitude and duration of the shear should be sufficient to reduce the size of the internal phase droplets to form stable emulsions, and ideally to reduce the size to below about 40 microns. Traditional liquid emulsions generally have a shear range tolerance of 6 m/s up to 25 m/s.

The glycerin internal phase, prior to addition to the external oil phase, is characterized as having a complex viscosity of less than 2 Pa·s when subjected to an oscillatory stress of 100 Pa during an oscillatory stress sweep conducted at a temperature of 25° C. and an angular frequency of 1 Hz. The complex viscosity, as used herein and in the following claims, may be measured on a TA G2 Stress Controlled Rheometer with a parallel plate geometry gap of 500 micron.

### Example 2

### Stability Test

Stability is monitored in high temperature (110 F) and alternating temperature conditions as well as freeze-thaw conditions over 28 days. Sample stability is rated on a 0-5 point scale in which a rating of 0 is given to a perfectly stable sample (e.g., no syneresis) and a rating of 5 is given to a sample showing large scale signs of instability (e.g., significant syneresis). Stability conditions were monitored in the following conditions over a 28 day period in each instance: (1) 110 F constant temperature; (2) 77 F constant temperature; (3) 40 F constant temperature; (4) 40 F/110 F alternating temperature; and (5) freeze-thaw. A rating was derived from the arithmetic average of the stability of a given material in each of the above conditions after 28 days.

### Example 3

### Effect of Water Content

To illustrate the improvement in stability as a result of increasing water content, iterations of the formula of the present invention were compounded and their stability over 4 weeks compared, using the stability test of Example 1 The stability results are shown in Table 2, showing that increased water concentration greatly improves stability.

TABLE 2
The effect of water concentration

| | Formula 1 | Formula 2 |
|---|---|---|
| Component | % | % |
| Oils and Waxes | 60.4% | 58.4% |
| Emulsifiers | 5.5% | 5.5% |
| Colorants | 12.7% | 12.7% |
| Glycerin | 20% | 20% |
| Distilled water | 1% | 3% |
| Fragrance | 0.4% | 0.4% |
| TOTAL | 100% | 100% |
| Stability rating | 4 | 2 |

Of course, rather than deliberately add water to the glycerin, the same effect may be achieved by using “wet” glycerin, by which is meant glycerin that has absorbed water from the air. It is within the skill in the art to determine the water content of the starting glycerin.

### Example 3

### Effect of Xanthan Gum

To illustrate the improvement in stability as a result of the addition of Xanthan gum, iterations of the formula of the present invention were compounded and their stability over 4 weeks compared. The stability results are shown in Table 3, whereby addition of Xanthan gum to the formula greatly improved the stability.

TABLE 3
The effect of Xanthan gum

| | Formula 5 | Formula 6 |
|---|---|---|
| Component | % | % |
| Waxes and Oils | 58.4% | 58.4% |
| Emulsifiers | 5.5% | 5.5% |
| Colorants | 12.7% | 12.7% |
| Glycerin | 20% | 20% |
| Distilled water | 2.8% | 3.0% |
| Xanthan Gum | 0.2% | 0 |
| Fragrance | 0.4% | 0.4% |
| TOTAL | 100% | 100% |
| Stability rating | 0 | 2 |

### Example 4

### Effect of Xanthan Gum Concentration and Electrolyte Addition

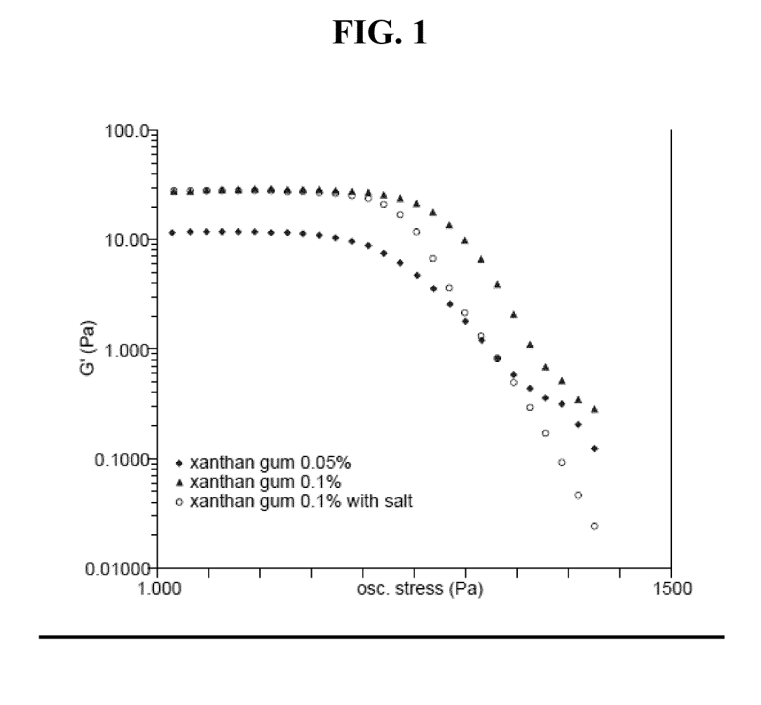

The storage modulus (G′) as a function of oscillating shear stress of the internal emulsion phase was tested, at different concentrations of Xanthan gum and with the addition of an electrolyte. As shown in FIG. 1, the addition of electrolyte at 0.1% reduces the yield point and shows more highly shear thinning behavior, which leads to better processability. A step down in Xanthan gum concentration (from 0.1% to 0.05%) decreased G′ overall, which leads to less storage stability. The addition of the electrolyte does not affect the unperturbed behavior of the internal emulsion phase.

### Example 5

### Formulations

Exemplary formulations of lip sticks according to the invention are provided below in Table 4.

TABLE 4

| | Ingredient Range |
|---|---|
| Formula Composition | % (w/w) |
| Waxes and Oils | 55-85 |
| Emulsifiers | 1-10 |
| Fragrance | 0-3 |
| Colorants | 5-20 |
| Glycerin | 10-30 |
| Water | 2-10 |
| Xanthan Gum | 0.01-1 |
| Salt | 0.01-1 |

In the formulation of Table 4, the salt is typically magnesium sulfate.

### Example 6

### Measurement of Pituitous Rheology

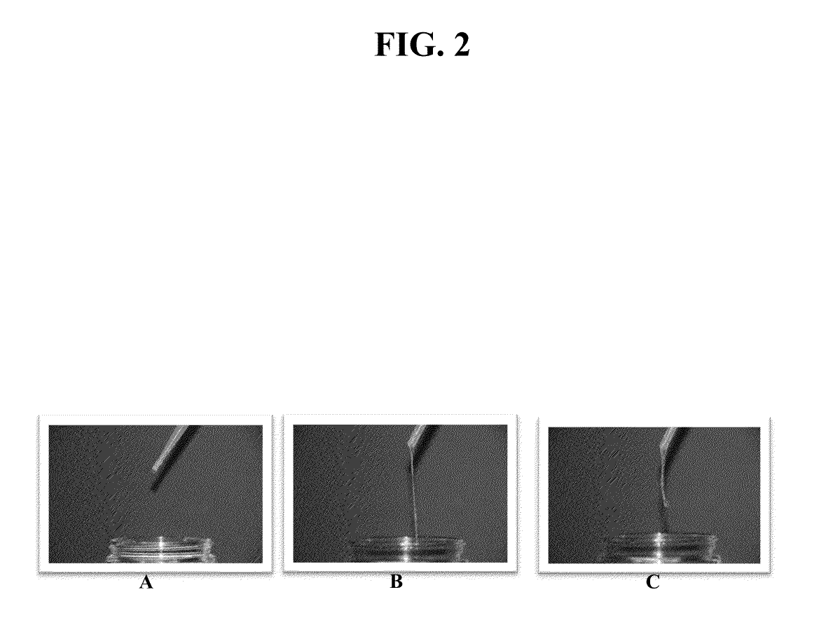

The following method may be used to characterize the pituitous rheology of the internal phase. The pituitous rheology manifests in an undesirable “stringiness” to the composition as shown in FIG. 2. A plastic pipette is dipped into the internal phase of the emulsion and held three inches above surface of the liquid for 20 seconds. When the internal emulsion phase consisting of glycerol and water lacks the polysaccharide thickener Xanthan gum, it shows no pituitous behavior, as evidenced by no measurable “string” of material connecting the pipette tip and the liquid seen in image “A” in FIG. 2. In contrast, when Xanthan gum is added to the internal phase, a string that does not break upon lifting the pipette from the solution is formed, as shown in image “B” in FIG. 2. When an electrolyte (magnesium sulfate) is added to the Xanthan gum-containing internal phase, the internal emulsion phase shows a decrease in pituitous rheology whereby the “string” of material breaks immediately upon removal of the pipette from the jar, as shown in image “C” in FIG. 2.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described therein will become apparent to those skilled in the art from the foregoing description. Such modifications are also intended to fall within the scope of the appended claims. All publications cited herein are incorporated by reference in their entirety.

1. A glycerin-in-oil emulsion comprising:
(i) a continuous phase comprising one or more topically-acceptable oils;
(ii) a discontinuous phase comprising
(a) glycerin;
(b) water; and
(c) an anionic polysaccharide;
wherein, said emulsion is characterized by improved stability compared to an otherwise identical emulsion not containing the anionic polysaccharide.

2. The emulsion of claim 1, wherein the emulsion is stable at 50° C. for at least four weeks.

3. The emulsion of claim 1, wherein the continuous phase further comprises a wax and the emulsion is a solid emulsion.

4. The emulsion of claim 1, wherein said water comprises from about 3% to about 14% by weight of the entire emulsion.

5. The emulsion of claim 1, wherein said water comprises from about 3% to about 5% by weight of the entire emulsion.

6. The emulsion of claim 1, wherein the polysaccharide comprises from about 0.01% to about 5% by weight of the entire emulsion.

7. The emulsion of claim 1, wherein the polysaccharide comprises from about 0.1% to about 1% by weight of the entire emulsion.

8. The emulsion of claim 1, wherein the anionic polysaccharide is Xanthan gum.

9. The emulsion of claim 1, wherein the discontinuous phase further comprises an electrolyte.

10. The emulsion of claim 9, wherein the electrolyte is magnesium sulfate.

11. The emulsion of claim 1, wherein the rheology of the discontinuous phase is characterized by a complex viscosity of less than 2 Pa·s when subjected to an oscillatory stress of 100 Pa during an oscillatory stress sweep conducted at a temperature of 25° C. and an angular frequency of 1 Hz.

12. The emulsion of claim 1, wherein the discontinuous phase has a maximum droplet size of 40 microns.

13. The emulsion of claim 13, wherein the discontinuous phase has a median droplet size of 1-10 microns.

14. A lip product comprising the emulsion of claim 1, selected from the group consisting of lip cream, lip balm, lip gloss, medicated lip treatment, lip moisturizer, lip cosmetic, lip sunscreen, and lip flavorant.

15. A glycerin-in-oil emulsion comprising:
(a) about 20 to about 90% by weight of one or more topically-acceptable oils;
(b) about 5% to about 65% by weight glycerin;
(c) about 3% to about 14% by weight water;
(d) about 0.01% to about 5% by weight of an anionic polysaccharide; and
(e) about 0.01 to about 3% by weight of an electrolyte; and
wherein, component (a) comprise a continuous phase and components (b)-(e) comprise a discontinuous phase of said glycerin-in-oil emulsion; and wherein said glycerin-in-oil emulsion is characterized by improved stability compared to an otherwise identical emulsion not containing the anionic polysaccharide; and wherein the maximum droplet size of the discontinuous phase is about 40 microns.

16. The emulsion of claim 15, wherein the electrolyte is magnesium sulfate.

17. The emulsion of claim 15, wherein the electrolyte is present at about 0.1-2% by weight of the emulsion.

18. The emulsion of claim 15, wherein the rheology of the discontinuous phase is characterized by a complex viscosity of less than 2 Pa·s

19. The emulsion of claim 15, further comprising, in the continuous phase, discontinuous phase, or both, from about 0.001% to about 30% by weight of additional ingredients selected from film forming polymers, emulsifiers, rheology modifiers, thickeners, stabilizers, dispersants, humectants, emollients, conditioners, active ingredients, antimicrobials, preservatives, antioxidants, pH adjusters, chelators, sequestering agents, colorants, cosmetic particulates fragrances, flavorants, and combinations thereof.

20. A method for making a glycerin-in-oil emulsion comprising:
(i) providing a continuous phase comprising one or more topically-acceptable oils;
(ii) providing a discontinuous phase comprising glycerin, water, a polysaccharide thickener, and an electrolyte; the discontinuous phase being characterized by a complex viscosity of less than 2 Pa·s when subjected to an oscillatory stress of 100 Pa during an oscillatory stress sweep conducted at a temperature of 25° C. and an angular frequency of 1 Hz; and
(iii) adding the discontinuous phase to the continuous phase while maintaining an amount of shear sufficient to control droplet size of the discontinuous phase to be no more than 40 microns.
